(19) United States
(12) Patent Application Publication
(10) Pub. No.: US 2025/0072996 A1
(43) Pub. Date: Mar. 6, 2025

(54) MANUFACTURING METHOD OF RETAINER

(71) Applicant: SMILECAD CO., LTD., Busan (KR)

(72) Inventors: Seok Joon KIM, Busan (KR); Jae Ho JEON, Busan (KR); Byeong Soo KIM, Busan (KR)

(73) Assignee: SMILECAD CO., LTD., Busan (KR)

(21) Appl. No.: 18/823,659

(22) Filed: Sep. 3, 2024

(30) Foreign Application Priority Data
Sep. 5, 2023 (KR) 10-2023-0117524

(51) Int. Cl. A61C 5/00; A61C 9/00; B23K 26/38

(52) U.S. Cl. CPC A61C 5/007; A61C 9/0053; B23K 26/38

(57) ABSTRACT

Proposed is a manufacturing method of a retainer provided with a body part having a first surface being in contact with a teeth surface of a plurality of teeth forming a teeth arrangement so as to support the teeth arrangement. The manufacturing method includes a process of acquiring three-dimensional scanning data of the teeth arrangement, a data processing process in which processing line data for manufacturing the body part are generated, and a laser cutting process in which a body forming part is manufactured from a base material formed of a shape memory alloy material. In the data processing process, tangential lines that extend along the teeth surface of the teeth arrangement are formed and collected, and a first processing line formed by connecting, to each other, tangential line portions in a tangential line group body and a second processing line spaced apart from the first processing line are generated.

### CROSS-REFERENCE TO RELATED APPLICATION

The present application claims priority to Korean Patent Application No. 10-2023-0117524, filed Sep. 5, 2023, the entire contents of which are incorporated herein for all purposes by this reference.

### BACKGROUND

### Field of the Invention

The present disclosure relates to a manufacturing method of a retainer. More particularly, the present disclosure relates to a manufacturing method of a retainer manufactured by performing laser cutting on a shape memory alloy material on the basis of three-dimensional scanning data of a teeth arrangement.

### Description of Related Art

A retainer is a type of a teeth arrangement maintenance device used to maintain a teeth arrangement after orthodontic treatment is finished and then an orthodontic device is removed.

Such a retainer may be largely divided into an adhesive retainer that is attached to the teeth and a removable retainer that is capable of being arbitrarily detached from the teeth like a mouthpiece.

Generally, the adhesive retainer is manufactured by a person bending a wire formed of a stainless steel material corresponding to a plaster model shape of the teeth that have been treated.

In addition, in the removable retainer, a retainer body part that is in contact with a front side surface of all teeth is manufactured by a person bending a wire formed of a stainless steel material corresponding to a plaster model shape of the teeth that have been treated, and the retainer body is manufactured such that the retainer body is in close contact with the palate by turning around each rear surface of the rearmost teeth. In addition, when the retainer body is manufactured, an acrylic resin portion that covers the palate is manufactured in a shape coupled to the retainer body part. After the retainer body is fixed to the plaster model shape, the acrylic resin portion is manufactured by alternately spraying a powder polymer and a liquid monomer on a palate portion so that the polymer and the monomer are mixed with each other.

Meanwhile, since the retainer manufactured by bending a conventional stainless steel material is easily deformed, there are problems that the teeth may be distorted, and there are problems that the retainer is difficult to be manufactured in a shape that is in complete contact with a plurality of teeth forming a teeth arrangement, so that a large shape deviation occurs.

Recently, a retainer to which a shape memory alloy is applied has been developed so as to prevent a deformation of a retainer to which a stainless steel material is applied and to improve the correction effect.

In this regard, in the Korean Patent No. 10-2350800 B1, a technology manufacturing a retainer in contact with a teeth arrangement by laser cutting a shape memory alloy material is disclosed.

Meanwhile, including the description of the related art, conventionally, a body part of a retainer following a teeth arrangement is designed on the basis of a teeth surface having a specific height of the teeth arrangement. In this case, due to the influence of the difference in inclination and flexure of the teeth at each position and due to the influence of a vertical height of the body part, it is difficult to manufacture the body part that is in complete close contact with a plurality of teeth immediately after performing laser cutting.

In detail, FIGS. 1A to 1C are views illustrating a partial interference state and a no contact state between a body part of a retainer generated by conventional laser cutting and teeth.

FIGS. 1A, 1B, and 1C are views illustrating an example illustrating differences in inclination and flexure at each position of teeth forming a teeth arrangement, a state in which interference between a laser cutting surface of a body part 6 having a predetermined vertical height and a teeth surface due to the differences, and a no contact state.

As illustrated in FIGS. 1A, 1B, and 1C, due to a rectilinear propagation of a laser, the cutting surface of the body part 6 in contact with the teeth surface of the teeth arrangement is formed vertically.

Conventionally, a planar shape of the body part 6 that follows the teeth arrangement is designed on the basis of the teeth surface having the specific height of the teeth arrangement, and a three-dimensional shape of the body part 6 is designed by setting a vertical height to the planar shape of the designed body part 6.

In this case, the body part 6 is capable of being in contact with the teeth arrangement without a gap only when a center portion of the height of the body part 6 that was the standard for planar design is in complete contact with the teeth surface that is the standard for the design.

However, the body part 6 immediately after the laser cutting is performed may be partially interfered with the teeth surface due to the vertical height, or may be not in contact with the teeth surface and become loose. Referring to FIGS. 1A, 1B, and 1C, in parts in FIGS. 1A and 1B, a center portion of the body part 6 is not capable of being in contact with the teeth surface due to the interference and, accordingly, the body part 6 is not capable of being in contact with the teeth surface and may be in a separated state in a part in FIG. 1C. In order to solve such problems, additional shape processing or design changes are required to be performed.

Therefore, it is difficult to easily manufacture a retainer that is in stable contact with teeth at a required position, and there is a problem that manufacturing time and cost are significantly increased as additional processing or design changes is required.

### DOCUMENT OF RELATED ART

(Patent Document 1) Korean Patent No. 10-2350800 B1

### SUMMARY OF THE INVENTION

Accordingly, the present disclosure has been made keeping in mind the above problems occurring in the related art, and an objective of the present disclosure is to provide a manufacturing method of a retainer capable of being in stable contact with a plurality of teeth forming a teeth arrangement.

Another objective of the present disclosure is to provide a manufacturing method of a retainer formed as a single integrated configuration from a shape memory alloy material.

In order to achieve the objectives of the present disclosure, according to the present disclosure, there is provided a manufacturing method of a retainer provided with a body part which has a curved shape along a teeth arrangement and which has a first side configured to be in contact with a teeth surface of a plurality of teeth forming the teeth arrangement so as to support the teeth arrangement, the manufacturing method including: a process of acquiring three-dimensional scanning data of the teeth arrangement by using a three-dimensional scanning device; a data processing process in which processing line data defining a laser cutting shape for manufacturing the body part are generated on the basis of the teeth arrangement on the scanning data; and a laser cutting process in which a body forming part configuring the body part is manufactured by laser cutting a base material formed of a shape memory alloy material on the basis of the generated processing line data, wherein the data processing process includes: a plurality of tangential lines formation process in which a plurality of tangential lines that extends along the teeth surface of the teeth arrangement at different heights is formed; a tangential line collection process in which a tangential line group body is generated by disposing the plurality of tangential lines on a same plane; and a processing line generation process in which a first processing line formed by connecting, to each other, tangential line portions in the tangential line group body that are positioned furthest from a side where the teeth arrangement is positioned and a second processing line that is spaced apart from the first processing line toward a direction away from the side where the teeth arrangement is positioned so as to define a front-to-rear surface of the body forming part are generated.

In addition, in the plurality of tangential lines formation process, a plurality of planes which traverses the teeth arrangement and which is spaced vertically apart from each other may be generated, and the plurality of tangential lines may be generated by extracting a contact surface between the teeth surface of the teeth arrangement and the plurality of planes.

In addition, in the plurality of tangential lines formation process, the plurality of tangential lines may be generated within a range of a vertical height of the body part that is required.

In addition, the plurality of tangential lines may include: an upper tangential line in contact with the teeth surface of the teeth arrangement at a height corresponding to an upper end of a virtual body part; a lower tangential line in contact with the teeth surface of the teeth arrangement at a height corresponding to a lower end of the virtual body part; and a middle tangential line in contact with the teeth surface of the teeth arrangement at a height between the upper tangential line and the lower tangential line.

In addition, the middle tangential line may be formed as a plurality of middle tangential lines spaced vertically apart from each other at a height between the upper tangential line and the lower tangential line.

In the manufacturing method of the retainer according to an embodiment of the present disclosure, following effects may be realized.

In manufacturing, by laser cutting, the body part of the retainer that is in contact with and supporting the plurality of teeth forming the teeth arrangement, the processing line data defining the laser cutting shape considers the vertical height of the body part, and is designed on the basis of the plurality of tangential lines that extend along the teeth surface of the teeth arrangement at different heights. In detail, the processing line data are generated by including the processing line connecting the tangential line portions that are positioned furthest from the center of the teeth arrangement in the tangential line group body in which the plurality of tangential lines is collected so as to overlap with each other.

Therefore, the cutting surface of the body part that is laser cut on the basis of the processing line data may have a shape that is in contact with the teeth surface along the teeth arrangement without a gap regardless of the differences in the inclination of the teeth surface at each position of the teeth and in the flexure of the teeth surface at each position of the teeth. That is, without any additional processing, the body part is capable of being formed such that the body part has the shape that is in contact with all teeth forming the teeth arrangement without a gap. According to the present disclosure, the manufacturing of the retainer becomes easier, the manufacturing time may be significantly reduced, and the treatment effect may be significantly improved by stably supporting the teeth arrangement by the retainer being in stable contact with the teeth at a position where the manufactured retainer is required.

To make the aforementioned more comprehensible, several embodiments accompanied with drawings are described in detail as follows.

### BRIEF DESCRIPTION OF THE DRAWINGS

The above and other objectives, features, and other advantages of the present disclosure will be more clearly understood from the following detailed description when taken in conjunction with the accompanying drawings, in which:

FIGS. 1A to 1C are views illustrating a partial interference state and a no contact state between a body part of a retainer generated by conventional laser cutting and teeth;

FIG. 2 is a view illustrating a state in which a retainer according to an embodiment of the present disclosure is attached to the teeth;

FIG. 3 is a plan view illustrating the retainer according to an embodiment of the present disclosure;

FIGS. 4A to 4D are views illustrating contact states between the retainer according to an embodiment of the present disclosure and the teeth for each position;

FIG. 5 is a view schematically illustrating a manufacturing process of the body part according to an embodiment of the present disclosure;

FIG. 6 is a flowchart illustrating a manufacturing method of a retainer according to an embodiment of the present disclosure;

FIG. 7 is a flowchart specifically illustrating a data processing process according to an embodiment of the present disclosure;

FIG. 8 and FIG. 9 are views illustrating a plurality of tangential lines formation process according to an embodiment of the present disclosure;

FIG. 10 is a view illustrating a plurality of tangential lines generated in the plurality of tangential lines formation process according to an embodiment of the present disclosure;

FIG. 11 is a view illustrating a tangential line collection process according to an embodiment of the present disclosure; and

FIG. 12 and FIG. 13 are views illustrating a processing line generation process according to an embodiment of the present disclosure.

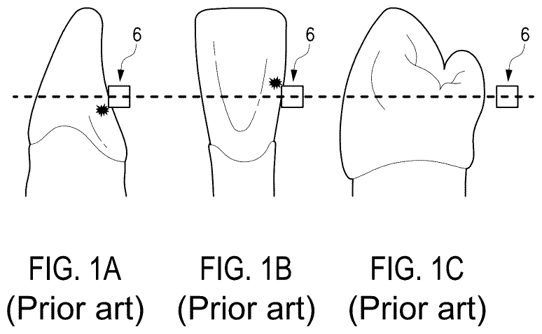

### DESCRIPTION OF THE EMBODIMENTS

Hereinafter, an embodiment of the present disclosure will be described in detail with reference to the accompanying drawings.

However, the spirit of the present disclosure is not limited to the presented embodiment, and by addition, modification, and removal of another component, other embodiments included within other degenerative inventions or the scope of the spirit of the present disclosure may be easily proposed.

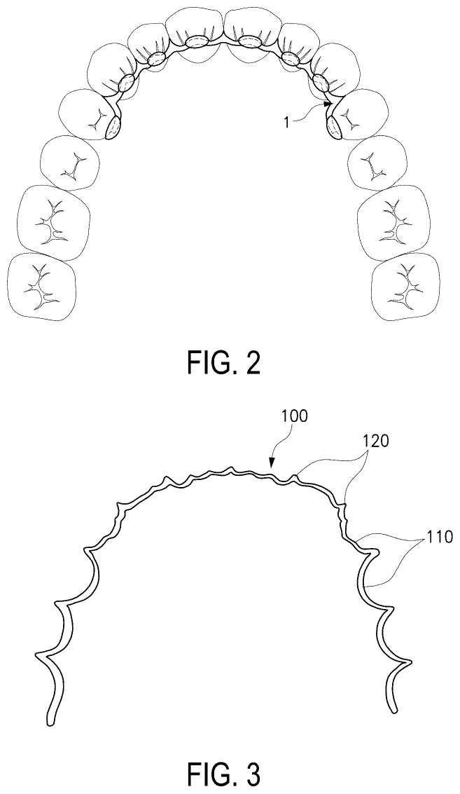

FIG. 2 is a view illustrating a state in which a retainer according to an embodiment of the present disclosure is attached to the teeth.

FIG. 3 is a plan view illustrating the retainer according to an embodiment of the present disclosure.

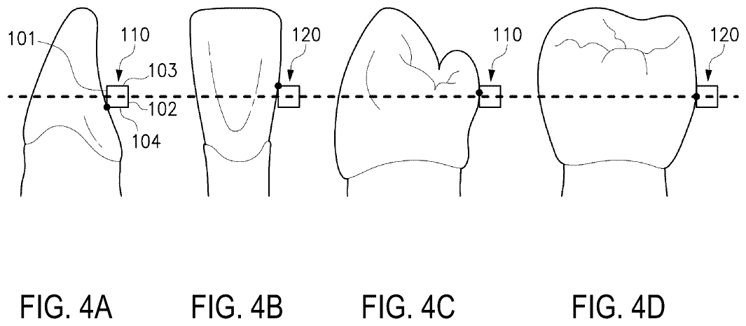

FIGS. 4A to 4D are views illustrating contact states between the retainer according to an embodiment of the present disclosure and the teeth for each position.

A manufacturing method S1 of a retainer of the present disclosure may be applied to a removable retainer, or may be applied to an adhesive retainer.

A retainer 1 manufactured by the manufacturing method S1 of the retainer of the present disclosure may include a body part 100 having a curved shape corresponding to an arrangement of human teeth.

When the body part 100 is formed such that the body part 100 is in contact with the teeth from a front side of the teeth arrangement and a resin region covering the palate is coupled to and applied to the body part 100, the removable retainer detachably coupled to the teeth arrangement may be realized.

On the other hand, when the body part 100 is applied such that the body part 100 is in contact with the teeth from the front side or a rear side of the teeth arrangement and is attached by an adhesive material, the adhesive retainer may be realized.

Hereinafter, the adhesive retainer which is in contact with a rear surface of the teeth from the rear side of the teeth arrangement on a lingual side and which is attached by an adhesive material will be described as an example of the retainer 1, and will be described in detail.

The body part 100 extends along the teeth arrangement formed by a plurality of teeth, and is formed such that the body part 100 reflects a transverse flexure of a teeth surface of the plurality of teeth, so that the body part 100 may be formed such that the body part 100 is completely in contact with the teeth surface along the teeth arrangement without a gap.

In addition, the body part 100 may be formed of a shape memory alloy material, and the body part 100 may be formed such that the body part 100 remembers a shape that is in contact with the teeth surface along the teeth arrangement without the gap.

Nitinol may be applied as the shape memory alloy material, and the shape memory alloy material may be applied such that the shape memory alloy material has a characteristic of returning the shape to the remembered shape from a temperature atmosphere in the oral cavity.

Therefore, the teeth arrangement may be stably maintained by the body part 100. Furthermore, even when the teeth arrangement is disarranged while the body part 100 is attached to the teeth arrangement, the teeth arrangement may be restored to an initial even teeth arrangement state by a shape restoration force of the body part 100.

More specifically, the body part 100 may be formed such that the body part 100 has a curved shape when the body part 100 is viewed from above, and may be formed such that the body part 100 has an approximately bow shape. In addition, the body part 100 may be formed such that the body part 100 has a predetermined vertical height and a predetermined front-to-rear thickness.

At this time, the front-to-rear thickness of the body part 100 refers to a distance between a surface facing the teeth from each portion along a curved longitudinal direction and a surface opposite to the surface. That is, the front-to-rear thickness of the body part 100 may be viewed as referring to a distance between a surface facing the buccal side and a surface facing the lingual side in the oral cavity.

The body part 100 may be formed such that the body part 100 has: a front surface 101 attached to the teeth surface; a rear surface 102 positioned opposite to the front surface 101; an upper surface 103 that connects an upper end of the front surface 101 and an upper end of the rear surface 102 to each other; and a lower surface 104 that connects a lower end of the front surface 101 and a lower end of the rear surface 102 to each other.

Referring to FIG. 2 and FIG. 3, the body part 100 may include at least one curved protrusion part 120 which is inserted into an interdental space formed between teeth adjacent to each other and which is in contact with inner adjacent surfaces of the teeth, and may include at least one support part 110 in contact with the teeth surface of each of teeth outside the interdental space.

The curved protrusion part 120 may be defined as a part of the body part 100, the part of the body part 100 protruding toward the interdental space such that the curved protrusion part 120 is inserted into the interdental space and is in close contact with side surfaces of the teeth.

When a plurality of curved protrusion parts 120 is formed, the plurality of curved protrusion parts 120 may be considered to be connected to each other by the support part 110.

Meanwhile, the fact that the body part 100 is formed such that the body part 100 is in complete contact with the teeth surface along the teeth arrangement without the gap may be described such that the front surface of the body part 100 is in point contact with or line contact with the teeth surface of the teeth at each portion along an extension direction thereof.

In addition, the body part 100 may be formed by performing laser cutting on the shape memory alloy material having a flat plate shape. The front surface 101 and the rear surface 102 may be cut surfaces generated by the laser cutting. In this case, the rectilinear propagation of the laser allows the cut surface at longitudinal cross-sectional surface of the body part 100 at each portion in the extension direction may be formed in a substantially rectangular surface shape, and the longitudinal cross-sectional surface of the body part 100 may be formed such that a front side facing the teeth surface has a vertical shape.

In addition, each tooth forming the teeth arrangement may have various teeth surface shapes, and a longitudinal cross-sectional surface of the teeth at each portion may have a shape having a rear side facing the body part 100 is curved or inclined or may have a vertical shape.

Therefore, the longitudinal cross-sectional surface of the body part 100 may be in point contact with or in line contact with the longitudinal cross-sectional surface of the teeth according to the shape of the longitudinal cross-sectional surface of the teeth corresponding to each portion.

Hereinafter, a contact state between the body part 100 and the teeth according to the cross-sectional shape of the teeth will be described in detail with reference to FIG. 4A to FIG. 4D as an example.

FIG. 4A is a view illustrating a state in which a lingual side tooth surface of a first tooth forming the teeth arrangement and the support part 110 corresponding to the lingual side tooth surface of the first tooth are in contact with each other.

FIG. 4B is a view illustrating a state in which a side surface of the first tooth and the curved protrusion part 120 corresponding to the side surface of the first tooth are in contact with each other.

FIG. 4C is a view illustrating a state in which a lingual side tooth surface of a second tooth forming the teeth arrangement and the support part 110 corresponding to the lingual side tooth surface of the second tooth are in contact with each other.

FIG. 4D is a view illustrating a state in which a side surface of the second tooth and the curved protrusion part 120 corresponding to the side surface of the second tooth are in contact with each other.

As illustrated in FIG. 4A, the lingual tooth surface of the first tooth may have a shape that is inclined downward toward the support part 110, and a lower end portion of a front surface of the support part 110 corresponding to the lingual tooth surface of the first tooth may be in contact with the tooth surface.

As illustrated in FIG. 4B, the side surface of the first tooth may have a shape that is inclined upward toward the curved protrusion part 120, and an upper end portion of a front surface of the curved protrusion part 120 corresponding to the side surface of the first tooth may be in contact with the tooth surface.

As illustrated in FIG. 4C, the lingual tooth surface of the second tooth may have a shape that is inclined upward toward the support part 110, and an upper end portion of the front surface of the support part 110 corresponding to the lingual tooth surface of the second tooth may be in contact with the tooth surface.

As illustrated in FIG. 4D, the side surface of the second tooth may have a shape having a center portion that protrudes toward the curved protrusion part 120, and a center portion of the front surface of the curved protrusion part 120 corresponding to the side surface of the second tooth may be in contact with the tooth surface.

Meanwhile, the body part 100 may be formed by performing the laser cutting such that the front surface 101 and the rear surface 102 are parallel to each other and are formed vertically. In addition, the upper surface 103 and the lower surface 104 of the body part 100 may be formed by upper and lower surfaces of a material having a flat plate shape such that the upper surface 103 and the lower surface 104 are parallel to each other, and may be formed in horizontal planar shapes.

Meanwhile, cutting, polishing, and so on may be performed on corner parts of the body part 100 such that the corner parts of the body part 100 have rounded shapes.

Meanwhile, the curved protrusion part 120 may be formed in a shape that generally continues smoothly so that there are no sharp corners and valleys when the curved protrusion part 120 is viewed from above.

As an example, the curved protrusion part 120 may be formed such that an end portion that protrudes from the front surface of the curved protrusion part 120 is rounded. In addition, the curved protrusion part 120 may be formed such that an end portion recessed from the rear surface of the curved protrusion part 120 is rounded.

Therefore, occurrence of a fracture of the curved protrusion part 120 that has a bent shape may be effectively prevented.

In addition, the curved protrusion part 120 may be formed such that a front-to-rear thickness of the curved protrusion part 120 is thicker than a front-to-rear thickness of the support part 110.

In addition, the curved protrusion part 120 may be formed such that the front-to-rear thickness of the curved protrusion part 120 is gradually decreased so as to correspond to the front-to-rear thickness of the support part 110 as the curved protrusion part 120 approaches the support part 110.

Therefore, due to a reinforcement of the front-to-rear thickness of the curved protrusion part 120, occurrence of fracture of the curved protrusion part 120 may be more effectively prevented, and a deformation of the body part 100 may be effectively prevented.

Meanwhile, the body part 100 may be manufactured by performing additional processing for shape stabilizing of a shape memory alloy material after the laser cutting is performed on the shape memory alloy material.

In addition, the body part 100 may be manufactured by performing a thermal treatment for remembering the shape again after the laser cutting is performed on the shape memory alloy material.

In detail, the remembered shape of the body part 100 may become unstable due to the temperature of a laser beam when the laser cutting is performed on the body part 100. Therefore, the thermal treatment for remembering the shape again is performed after the body part 100 that is cut is fixed in the cut shape as is, the shape may be stabilized to a required shape.

Generally, the thermal treatment for the shape memory alloy material to remember the shape may be performed for 10 minutes to 1 hour at 300 degrees Celsius to 700 degrees Celsius.

In addition, the body part 100 may be manufactured by polishing a surface of the body part 100. Therefore, the body part 100 may be formed such that the body part 100 has a smoother surface and a sharp portion of the body part 100 is removed, so that the body part 100 may be formed in a generally safe structure.

Hereinafter, a manufacturing method of a retainer according to an embodiment of the present disclosure will be described in detail with reference to the drawings.

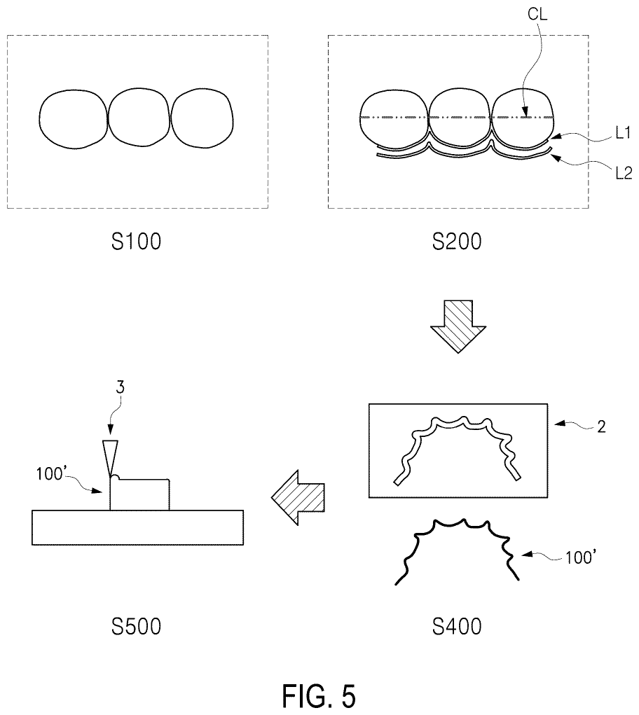

FIG. 5 is a view schematically illustrating a manufacturing process of the body part according to an embodiment of the present disclosure.

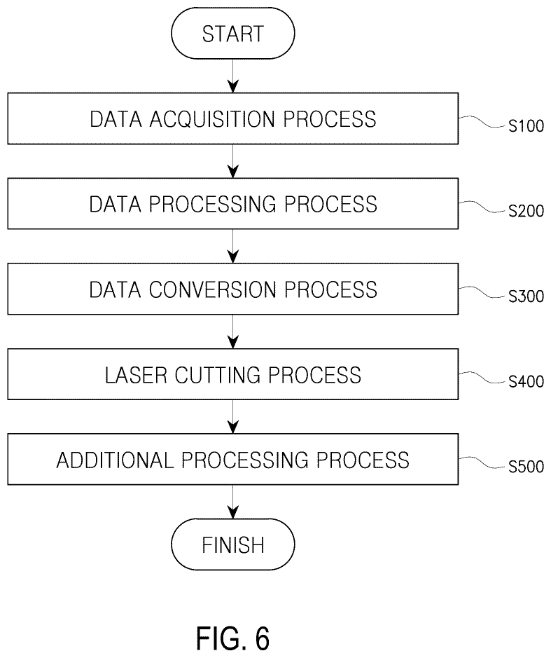

FIG. 6 is a flowchart illustrating the manufacturing method of the retainer according to an embodiment of the present disclosure.

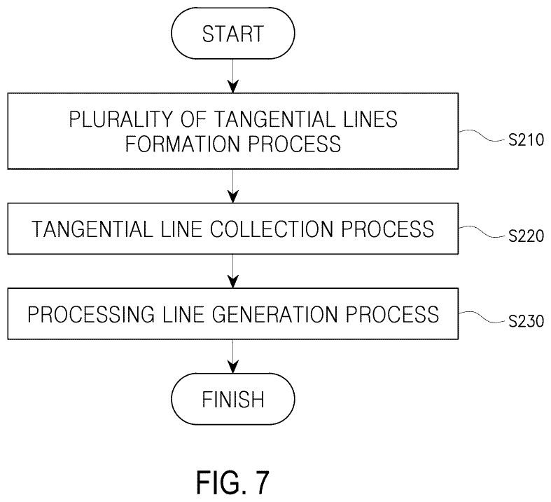

FIG. 7 is a flowchart specifically illustrating a data processing process according to an embodiment of the present disclosure.

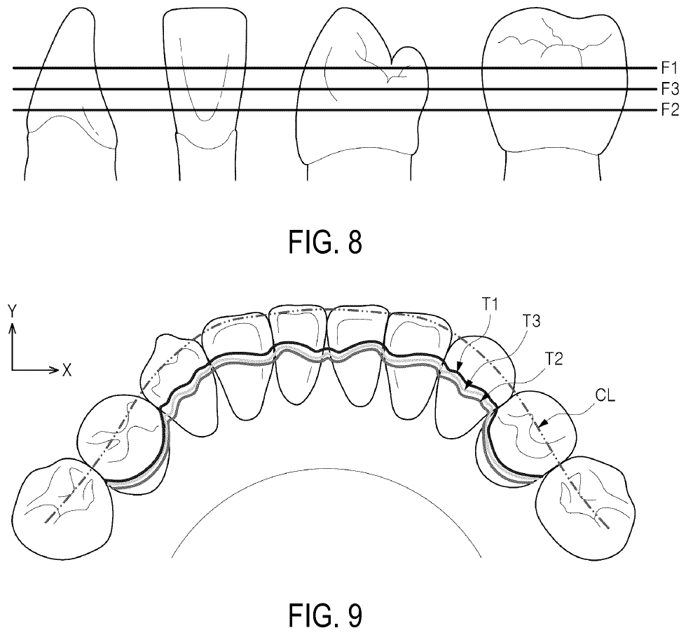

FIG. 8 and FIG. 9 are views illustrating a plurality of tangential lines formation process according to an embodiment of the present disclosure.

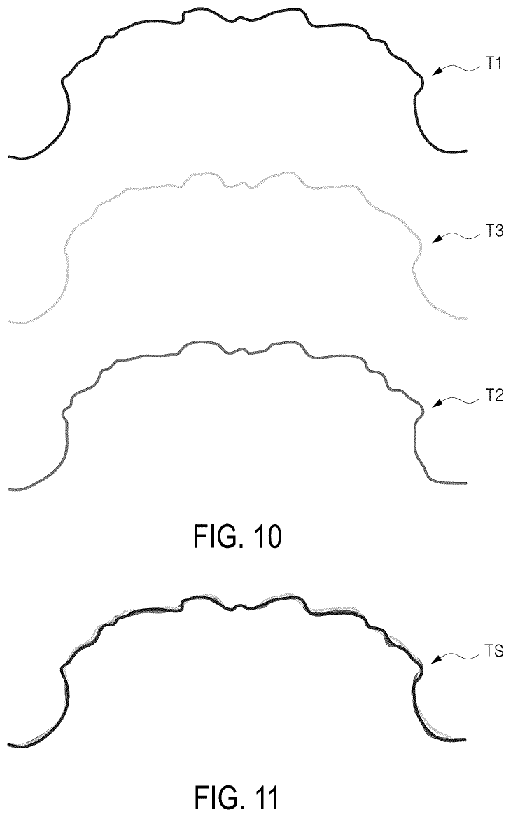

FIG. 10 is a view illustrating a plurality of tangential lines generated in the plurality of tangential lines formation process according to an embodiment of the present disclosure.

FIG. 11 is a view illustrating a tangential line collection process according to an embodiment of the present disclosure.

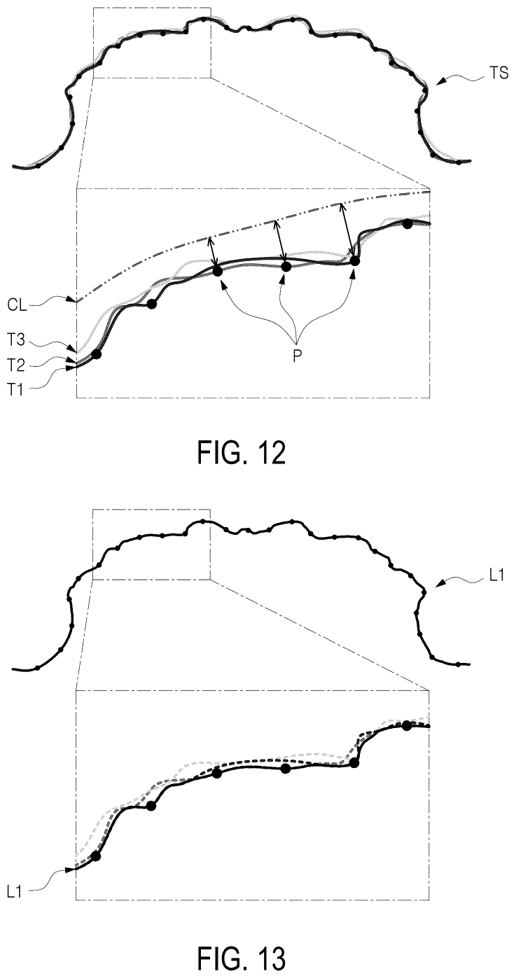

FIG. 12 and FIG. 13 are views illustrating a processing line generation process according to an embodiment of the present disclosure.

A manufacturing method S1 of a retainer according to an embodiment of the present disclosure may include a data acquisition process S100 in which scanning data of the teeth arrangement is acquired, a data processing process S200 in which processing line data for laser cutting is generated on the basis of the scanning data, a data conversion process S300 in which the processing line data that is generated is converted into data for laser processing, a laser cutting process S400 in which a base material is laser cut, and an additional processing process S500 in which a body forming part that is laser cut is additionally processed.

In the data acquisition process S100, three-dimensional scanning data of the teeth arrangement may be acquired by using a three-dimensional scanning apparatus.

At this time, scanning data refer to photographed data of the teeth arrangement of the upper jaw or the lower jaw where the body part 100 will be attached, the photographed data being photographed by using a 3D camera.

The scanning data may include the teeth arrangement, a shape of an interdental space shape formed in the teeth arrangement, a tooth surface curve of each tooth, a tooth surface inclination of each tooth, and so on.

The scanning data may be a file corresponding to an stl formatted file.

After the data acquisition process S100 is performed, the data processing process S200 for generating processing line data defining a laser cutting shape for manufacturing the body part 100 on the basis of the scanning data may be performed.

The data processing process S200 may be performed by using a computational design software that automatically generates a three-dimensional model including surface shape information and coordinate information on the basis of three-dimensional information input from the three-dimensional scanning apparatus.

The design software may be a design software configured such that a plane on a three-dimensional model is capable of being generated and a tangential line between the generated plane and a surface of the three-dimensional model is capable of being automatically or manually generated on the basis of the concepts of Computer-Aided Design (CAD) and Computer-Aided Manufacturing (CAM).

The data processing process S200 may include a plurality of tangential lines formation process S210, a tangential line collection process S220, a processing line generation process S230, and a processing line modification process S240.

In the plurality of tangential line formation process S210, a plurality of planes that traverse a plurality of teeth forming the teeth arrangement may be generated on the scanning data.

The plurality of planes may be horizontal planes, and the plurality of planes may be generated as a plurality planes spaced vertically apart from each other.

For example, a plane traversing the plurality of teeth forming the teeth arrangement may be defined as an XY coordinate plane, and the plurality of planes may be formed in an X-axis, a Y-axis, and a Z-axis perpendicular spaced apart by a Z-axis that is perpendicular to the X-axis and the Y-axis.

When it is assumed that a virtual body part is disposed at a required position on the teeth arrangement on the scanning data, the plurality of planes may include an upper plane F1 traversing the teeth arrangement at a height corresponding to an upper end of the virtual body part, a lower plane F2 traversing the teeth arrangement at a height corresponding to a lower end of the virtual body part, and a middle plane F3 traversing the teeth arrangement between the upper plane F1 and the lower plane F2.

A vertical thickness of the body part 100 that is manufactured may be set by a separation height between the upper plane F1 and the lower plane F2.

The middle plane F3 may be formed as a plurality of middle planes F3 between the upper plane F1 and the lower plane F3, and the plurality of middle planes F3 may be formed such that the plurality of middle planes F3 is spaced apart from each other vertically.

After the planes are generated on the scanning data, each tangential line between the surface of the teeth arrangement and the planes may be generated.

The tangential line may also be generated by a worker's drafting using the design software.

Alternatively, tangential line coordinates may be automatically extracted and automatically generated by the design software, and the shape may be corrected by modification work of a worker, and then the final shape may be generated.

The generated tangential line may be formed such that the tangential line is in full contact with the teeth surface along the teeth arrangement by reflecting the transverse flexure of the teeth surface of the teeth and the interdental shape at a formation height of the corresponding plane.

The tangential line may be formed in a shape similar to the shape of the body part 100 so that the shape of the tangential line is curved and has a protrusion part so as to be inserted into the interdental space, and may be formed continuously without disconnection.

The tangential line may be generated as a plurality of tangential lines corresponding to the number of planes.

The plurality of tangential lines may include an upper tangential line T1 defined as a tangential line between the teeth arrangement and the upper plane F1, a lower tangential line T2 defined as a tangential line between the teeth arrangement and the lower plane F2, and a middle tangential line T3 defined as a tangential line between the teeth arrangement and the middle plane F3.

The upper tangential line T1 may be in contact with the teeth arrangement at a height corresponding to the upper end of the virtual body part.

The lower tangential line T2 may be in contact with the teeth arrangement at a height corresponding to the lower end of the virtual body part.

The middle tangential line T3 may be in contact with the teeth arrangement at a height between the upper tangential line T1 and the lower tangential line T2.

After the plurality of tangential line formation process S210 is performed, the tangential line collection process S220 may be performed.

In the tangential line collection process S220, the plurality of generated tangential lines may be collected on a single XY plane, and may form a tangential line group body TS.

The plurality of tangential lines may have different shapes according to differences in the teeth surface shapes and the interdental space shapes of the teeth at each height. Therefore, the tangential line group body TS may have a twisted shape in which the plurality of tangential lines having different shapes intersects with each other along the teeth arrangement.

After the tangential line collection process S220 is performed, the processing line generation process S230 may be performed.

In the processing line generation process S230, a first processing line L1 for generating the front surface 101 of the body part 100 facing the teeth surface and a second processing line L2 for generating the rear surface 102 of the body part 100 by being spaced apart from the first processing line L1 to a side away from the teeth surface may be generated.

The first processing line L1 may be viewed as representing a laser cutting path for generating the front surface 101 of the body part 100.

The first processing line L1 may be generated by connecting the maximum separation points P along the teeth arrangement in the tangential line group body TS, the maximum separation points P being positioned furthest from the side where the teeth arrangement is located.

In detail, the maximum separation points P may be points furthest from a teeth arrangement centerline CL along the teeth arrangement in the tangential line group body TS.

More specifically, the maximum separation points P may be contact points that are positioned furthest from the teeth arrangement center line CL among the contact points between rectilinear lines perpendicular to the teeth arrangement centerline CL and the tangential line group body TS along the teeth arrangement, and the first processing line L1 may be formed of a group of the maximum separation points P.

The teeth arrangement centerline CL may be defined as a line connecting anterior-posterior thickness centers of an occlusal surface of each tooth along the teeth arrangement when the teeth arrangement is viewed from above. That is, the teeth arrangement centerline CL may be defined as a line connecting the anterior-posterior thickness centers of the occlusal surface of each tooth on the XY coordinate plane.

That is, in an embodiment of the present disclosure, it may be seen that the first processing line L1 is generated by connecting the portions of the tangential lines positioned at the rearmost position along the teeth arrangement in the tangential line group body TS.

The first processing line L1 may be generated such that the portions of the tangential lines positioned at the rearmost position is automatically extracted by the design software on the basis of the plurality of tangential lines and the coordinate information of the three-dimensional model of the teeth arrangement, and may be generated by the worker's drawing using the design software.

The second processing line L2 may be viewed as representing a laser cutting path for generating the rear surface 102 of the body part 100.

The second processing line L2 may be formed in a shape corresponding to the shape of the first processing line L1.

The second processing line L2 may be formed on the XY plane by being spaced apart rearward from the first processing line L1 by a predetermined distance, and an approximate front-to-rear thickness of the body part 100 may be set by a separation distance between the first processing line L1 and the second processing line L2.

That is, the second processing line L2 is spaced apart from the first processing line L1 toward a direction away from the teeth arrangement centerline CL, thereby being capable of defining the front-to-rear thickness of the body part 100 that is laser cut.

Meanwhile, in each of the first processing line L1 and the second processing line L2, a portion corresponding to the curved protrusion part 120 may be formed such that a protruding end portion is roundly processed. That is, a curved end portion of each of the first processing line L1 and the second processing line L2 may be processed such that the curved end portion is not sharp.

In addition, the first processing line L1 and the second processing line L2 may be processed such that a portion of each of the first processing line L1 and the second processing line L2 corresponding to the curved protrusion part 120 is spaced further apart from a portion of each of the first processing line L1 and the second processing line L2 corresponding to the support part 110. Therefore, the curved protrusion part 120 may be formed such that the front-to-rear thickness of the curved protrusion part 120 is thicker than the front-to-rear thickness of the support part 110.

Meanwhile, after the data processing process S200 is completed, the data conversion process S300 may be performed.

In the data conversion process S300, processing line data including data of the generated first processing line and data of the generated second processing line may be converted into laser processing data.

For example, the processing line data may be stored as two-dimensional data.

In addition, by using a program automatically generating a movement path of a laser cutting device on the basis of the two-dimensional data, the processing line data may be converted into the laser processing data.

The generated laser processing data may be a two-dimensional CAD file reflecting the processing line data.

After the data conversion process S300 is performed, the laser cutting process S400 may be performed.

In the laser cutting process S400, the laser processing data may be input into the laser cutting device for laser processing.

In addition, a base material 2 that is input may be laser cut as the laser cutting device is operated according to the laser processing data. That is, the laser cutting device may be regarded to perform the laser cutting on the base material 2 on the basis of the processing data.

Specifically, the base material 2 may be provided as a flat plate shape formed of a shape memory alloy material. That is, the base material 2 may be a plate material formed of a shape memory alloy material. For example, the base material 2 may be a thin plate of nitinol.

The base material 2 may be configured such that the base material 2 has a characteristic of returning to a remembered thin plate shape at the temperature in the oral cavity. For example, the base material 2 may be applied such that the base material 2 is returned to the remembered shape under a temperature condition of 30 degrees Celsius or more. Therefore, the body part 100 manufactured by cutting the base material 2 may be formed such that the body part 100 has a characteristic of returning to a cut shape at the temperature in the oral cavity.

The laser cutting device may be provided with a jig in which the base material 2 is seated and fixed. In addition, the base material 2 may be seated on and fixed to the jig.

A setting value may be input to the laser cutting device before performing laser cutting. For example, an operator may set setting values such as a processing speed of 200 mm/s, a processing acceleration of 3000 mm/s, a pulse-type pulse power of 300 w, a pulse frequency of 600 Hz, a pulse time of 400 us, an argon gas pressure of 3.0 mPa, and so on.

After the base material 2 is input and the setting value of the laser cutting device is set, the worker may operate the laser cutting device.

When the laser cutting device is operated, cutting of the base material 2 is performed according to the processing line data by the laser cutting device, and a body forming part 100′ having a basic shape of the body part 100 may be cut and formed in the base material 2.

After the laser cutting process S400 is performed, the additional processing process S500 may be performed.

In the additional processing process S500, processing for stabilizing the shape of the body forming part 100′ may be performed.

For example, in the additional processing process S500, thermal treatment processing for the shape of the body forming part 100′ to remember the laser cut shape again may be performed.

In detail, the remembered shape of the body forming part 100′ may become unstable due to the temperature of a laser beam when the laser cutting is performed on the body forming part 100′. Therefore, after the body forming part 100′ that is cut is fixed to a separate jig or the base material 2 as the cut shape is, thermal treatment for remembering the shape again is performed, so that the shape of the body forming part 100′ may be stabilized to the required shape.

Generally, the thermal treatment for the shape memory alloy material to remember the shape may be performed for 10 minutes to 1 hour at 300 degrees Celsius to 700 degrees Celsius.

In addition, in the additional processing process S500, mechanical processing or chemical processing may be performed so that the body forming part 100′ has a final shape of the body part 100.

For example, in the additional processing process S500, shape processing and polishing processing of the body forming part 100′ may be performed.

The shape processing may be performed by various tools, devices, and so on capable of cutting a surface of the body forming part 100′. For example, the shape processing may be performed by a bur 3 having a cone shape.

For example, by the shape processing, burrs remaining on a cutting portion of the body forming part 100′ may be removed.

In addition, by the shape processing, chamfering of corner portions of the body forming part 100′ may be realized.

The polishing processing may be performed by various tools, devices, and so on capable of polishing the surface of the body forming part 100′.

A method of polishing a shape memory alloy material is variously disclosed in a known technology, so that the detailed description thereof will be omitted.

The body part 100 manufactured by the polishing processing may have a smoother surface, and may have a generally safe structure since a sharp portion is removed.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A manufacturing method of a retainer provided with a body part which has a curved shape along a teeth arrangement and which has a first side configured to be in contact with a teeth surface of a plurality of teeth forming the teeth arrangement so as to support the teeth arrangement, the manufacturing method comprising:
a process of acquiring three-dimensional scanning data of the teeth arrangement by using a three-dimensional scanning device;
a data processing process in which processing line data defining a laser cutting shape for manufacturing the body part are generated on the basis of the teeth arrangement on the scanning data; and
a laser cutting process in which a body forming part configuring the body part is manufactured by laser cutting a base material formed of a shape memory alloy material on the basis of the generated processing line data,
wherein the data processing process comprises:
a plurality of tangential lines formation process in which a plurality of tangential lines that extends along the teeth surface of the teeth arrangement at different heights is formed;
a tangential line collection process in which a tangential line group body is generated by disposing the plurality of tangential lines on a same plane; and
a processing line generation process in which a first processing line formed by connecting, to each other, tangential line portions in the tangential line group body that are positioned furthest from a side where the teeth arrangement is positioned and a second processing line that is spaced apart from the first processing line toward a direction away from the side where the teeth arrangement is positioned so as to define a front-to-rear surface of the body forming part are generated.

2. The manufacturing method of claim 1, wherein, in the plurality of tangential lines formation process, a plurality of planes which traverses the teeth arrangement and which is spaced vertically apart from each other is generated, and the plurality of tangential lines is generated by extracting a contact surface between the teeth surface of the teeth arrangement and the plurality of planes.

3. The manufacturing method of claim 1, wherein, in the plurality of tangential lines formation process, the plurality of tangential lines is generated within a range of a vertical height of the body part that is required.

4. The manufacturing method of claim 3, wherein the plurality of tangential lines comprises:
an upper tangential line in contact with the teeth surface of the teeth arrangement at a height corresponding to an upper end of a virtual body part;
a lower tangential line in contact with the teeth surface of the teeth arrangement at a height corresponding to a lower end of the virtual body part; and
a middle tangential line in contact with the teeth surface of the teeth arrangement at a height between the upper tangential line and the lower tangential line.

5. The manufacturing method of claim 4, wherein the middle tangential line is formed as a plurality of middle tangential lines spaced vertically apart from each other at a height between the upper tangential line and the lower tangential line.
